(19) United States
(12) Patent Application Publication
(10) Pub. No.: US 2009/0112480 A1
(43) Pub. Date: Apr. 30, 2009

(54) METHOD AND APPARATUS FOR CLUSTERING GENE EXPRESSION PROFILES BY USING GENE ONTOLOGY

(75) Inventors: Minho Kim, Daejeon-city (KR); Ho-Youl Jung, Daejeon-city (KR); Myunggeun Chung, Incheon-city (KR); Pora Kim, Gwangmyeong-city (KR); Soo-Jun Park, Seoul (KR); Seon-Hee Park, Daejeon-city (KR)

Correspondence Address: CANTOR COLBURN, LLP, 20 Church Street, 22nd Floor, Hartford, CT, 06103, US

(73) Assignee: Electronics and Telecommunications Research Institute, Daejeon-city (KR)

(21) Appl. No.: 12/053,315

(22) Filed: Mar. 21, 2008

(30) Foreign Application Priority Data
Mar. 21, 2007 (KR) 10-2007-0027795
Oct. 4, 2007 (KR) 10-2007-0099927

(51) Int. Cl. G01N 33/48

(52) U.S. Cl. 702/19

(57) ABSTRACT

Provided are a method and apparatus for clustering gene expression profiles by using the Gene Ontology (GO). The method includes: selecting one or more GO terms from a GO tree; receiving gene expression data sets; classifying the gene expression data sets into groups according to the GO terms; firstly clustering gene expression data belonging to each of the groups based on a similarity of the gene expression data; and secondly clustering the gene expression data sets by using the result of the first clustering as a seed.

### CROSS-REFERENCE TO RELATED PATENT APPLICATION

This application claims the benefit of Korean Patent Application No. 10-2007-0027795, filed on Mar. 21, 2007, and Korean Patent Application No. 10-2007-0099927, filed on Oct. 4, 2007, in the Korean Intellectual Property Office, the disclosure of which is incorporated herein in its entirety by reference.

### BACKGROUND OF THE INVENTION

1. Field of the Invention

The present invention relates to clustering of gene expression profiles, and more particularly, to a method and apparatus for clustering gene expression profiles by using the Gene Ontology (GO).

The present invention is derived from a study conducted by the Ministry of Information and Communication (MIC) of the Republic of Korea and the Institute for Information Technology Advancement (IITA) as one of a number of new growth engine core IT technology development projects (Assignment Number: 2006-S-007-02; Assignment Name: Ubiquitous Health Care Module System).

2. Description of the Related Art

Genes are expressed in response to specific stimuli. The amount of gene expression varies according to various stimuli (experimental conditions) and time variation. Data obtained by measuring the amount of gene expression by conducting a micro-array experiment is gene expression data, i.e., gene expression profiles.

It is known that genes having similar functions have similar expression patterns. Therefore, genes having similar expression profiles are clustered (i.e. grouped), so that a biological relationship of genes belonging to the same cluster (group) can be analogized. In more detail, from the cluster analysis, unknown functions of a gene can be inferred from the known functions of another genes belonging to the same cluster, and biological correlations between genes having similar expression patterns can be analogized.

Conventional technologies of dividing (clustering) gene expression profiles into subsets of genes having similar expression patterns are as follows:

Gene expression data sets are clustered by using a neural network algorithm that is referred to as a self-organizing map (SOM). The SOM is used to cluster the gene expression data sets by learning a connection network having weights between input nodes and output nodes. The SOM is used to allocate input data (gene expression profiles in the form of a vector) to the most similar cluster representative (that is randomly determined in the initial state), and re-calculate weights of the connection network so as to be best suited to the currently allocated data. That is, the SOM is a kind of winner-take-all neural network algorithm. This method is able to discover the phase relationship between clusters by allocating similar clusters to its neighbor. But, many input parameters such as the topology of the SOM need to be determined and the quality of its a clustering result depends on the input parameters. Furthermore, the initial cluster representatives should be determined accurately.

Determining seed genes for each cluster (i.e., cluster representative), has been a main drawback of conventional dividing-based clustering methods. It is more effectively treated. In more detail, in order to extract seed genes of each clusters singular value decomposition (SVD) is applied to gene expression data that is Gaussian transformation. This method does not need a process of determining complex initial input parameters unlike the conventional clustering algorithms. But, the number of initial seed genes still need to be determined. A wrong selection of the number of initial seed genes may dramatically deteriorate the quality of clustering result. Moreover, this method does not focus on the biological function but the mathematical similarity, which results in an unclear biological analysis for detected gene groups.

A clustering method takes into account genes in the Gene Ontology (GO), unlike the above methods. This method is able to analyze individual functions of each gene included in a cluster, and to concentrates on candidate genes. And thereby, it may reduce unnecessary processing time. However, since only genes whose correlation is greater than a predetermined reference level are selected, useful information included in other genes may be lost.

The conventional methods must determine complex parameters or initial cluster representatives that have a significant influence on the quality of clustering results. Or it uses a mathematical similarity only, causing an unclear analysis of a biological function. Move over, although an analysis of the biological function is used, some important information may be lost or its application is limited.

### SUMMARY OF THE INVENTION

The present invention provides a method and apparatus for detecting similar expression gene groups, which ensures reliability of clustering seeds that have a significant influence on clustering result, and effectively uses Gene Ontology (GO) terms as clustering seeds, thereby enhancing biological meaning and reliability of the clustering result and reducing information loss of the GO term seeds.

According to an aspect of the present invention, there is provided a method of clustering gene expression profiles comprising: selecting one or more Gene Ontology (GO) terms from a GO tree; receiving gene expression data sets; classifying the gene expression data sets into groups according to the GO terms; firstly clustering gene expression data belonging to each of the groups based on a similarity of the gene expression data; and secondly clustering the gene expression data sets by using the result of the first clustering as a seed.

According to another aspect of the present invention, there is provided an apparatus for clustering gene expression profiles comprising: a GO selection unit selecting one or more GO terms from a GO tree; a gene expression data input unit receiving gene expression data sets; a classification unit classifying the gene expression data sets into groups according to the GO terms; a first clustering unit firstly clustering gene expression data belonging to each of the selected groups based on a similarity of the gene expression data; and a second clustering unit secondly clustering the gene expression data sets by using the result of the first clustering as a seed.

### BRIEF DESCRIPTION OF THE DRAWINGS

The above and other features and advantages of the present invention will become more apparent by describing in detail exemplary embodiments thereof with reference to the attached drawings in which:

FIG. 1 is a flowchart illustrating a method of clustering gene expression profiles by using the Gene Ontology (GO), according to an embodiment of the present invention;

FIG. 2 is a flowchart illustrating a method of firstly clustering gene expression data sets, according to an embodiment of the present invention;

FIG. 3 is a flowchart illustrating a method of secondly clustering gene expression data sets according to an embodiment of the present invention;

FIG. 4 illustrates a gene expression profile according to another embodiment of the present invention;

FIG. 5 illustrates a GO tree according to an embodiment of the present invention;

FIG. 6 illustrates a similarity map according to an embodiment of the present invention; and

FIG. 7 is a block diagram of an apparatus for clustering gene expression profiles according to an embodiment of the present invention.

### DETAILED DESCRIPTION OF THE INVENTION

Hereinafter, the present invention will be described in detail by explaining embodiments of the invention with reference to the attached drawings.

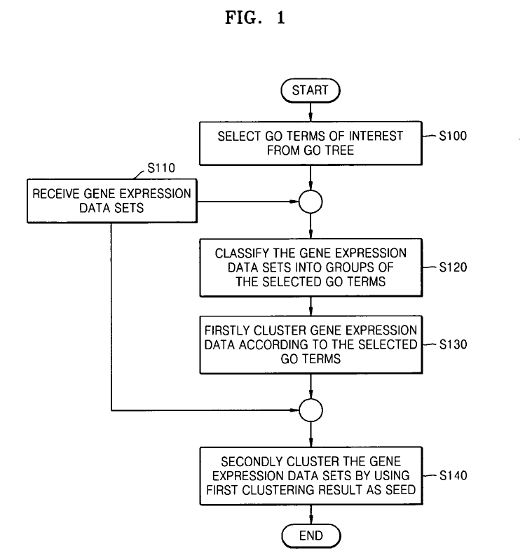

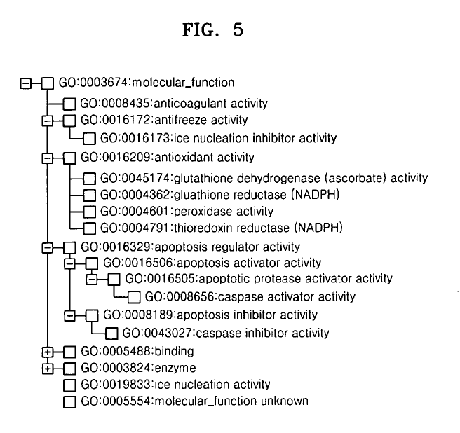

FIG. 1 is a flowchart illustrating a method of clustering gene expression profiles by using the Gene Ontology (GO), according to an embodiment of the present invention. Referring to FIG. 1, one or more GO terms of interest are selected from a GO tree (Operation 100). The GO has a tree structure in order to effectively represent relationships between GO terms. An example of the GO tree is illustrated in FIG. 5. A user may select the one or more GO terms of interest from the GO tree in a conventional manner by using a graphic user interface (GUI). The GO terms can be represented and selected using methods other than using the GUI.

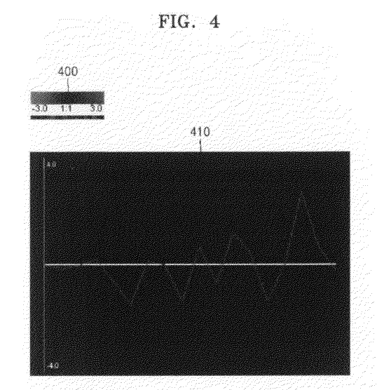

After the GO terms of interest are selected, gene expression data sets that are to be used for clustering are received (Operation 110). When a gene of a cell is exposed to specific conditions, the gene is expressed so as to create a material such as mRNA or DNA, i.e., a gene expression product. The specific conditions include exposure to a temperature, acidity (pH), growth/culture conditions, time variation, medicine or a candidate medicine material, etc. A value for measuring an amount of the gene expression product is a gene expression value. Expression values of a gene are gene expression profiles. An example of the gene expression profile is illustrated in FIG. 4. Referring to FIG. 4, an upper image 400 is a heat map having three colors, red, green, and black (RGB) according to expression values. A lower image 410 is a graph of expression values. Data sets with regard to gene expression profiles of each gene are the gene expression data sets of the present embodiment. It is obvious to one of ordinary skill in the art that the operation of inputting the gene expression data sets includes a preprocessing function, and thus the detailed description of the preprocessing function will not be provided.

After the GO terms of interest are selected and the gene expression data sets are inputted, the gene expression data sets are classified according to the selected GO terms of interest (Operation 120). Genes of the gene expression data sets have GO terms relating to their functions. That is, one gene can have a plurality of related GO terms. The genes are allocated to groups of the selected GO terms.

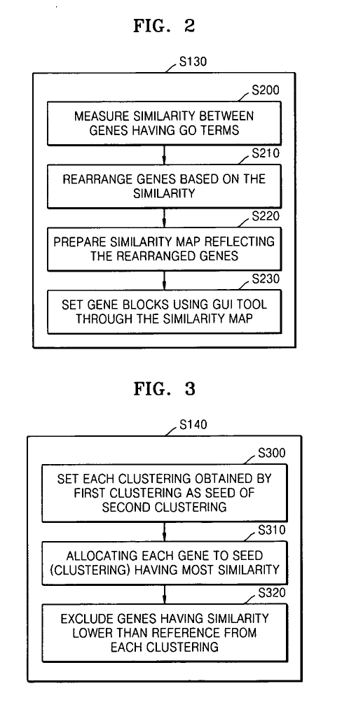

Thereafter, the gene expression data sets are firstly clustered according to is expression profile similarity of the genes allocated to each of the GO terms (Operation 130). The gene expression data sets are secondly clustered by using the result of the first clustering as a seed (Operation 140). The first and second clustering are described in detail with reference to FIGS. 2 and 3.

FIG. 2 is a flowchart illustrating a method of firstly clustering gene expression data sets, according to an embodiment of the present invention. Referring to FIG. 2, since the result of the first clustering is used as the seed of the second clustering, it is important to remove incorrect candidate seeds. Therefore, a conversational clustering method by a user is applied in the present embodiment. The first clustering is performed for each of the GO terms of interest.

A similarity between the gene expression profiles allocated to each of the GO terms of interest is calculated (Operation 200). The similarity is calculated using any one of the conventional methods. For example, a Pearson correlation coefficient is used to calculate the similarity. The similarity calculation is obvious to one of ordinary skill in the art and thus its detailed description will not be provided.

The genes are rearranged based on the similarity (Operation 210). In this regard, it is most important to sequentially extend the gene sets from any one of the genes to additional genes. The additional genes are the most similar to a currently created gene set. A similarity between the sets and the gene can be calculated using the conventional various methods. A sequence of extending the gene sets from any one of the genes to the additional genes is a sequence of the rearranged genes. The order of inclusion of the gene in expanding the set is that of rearrangement.

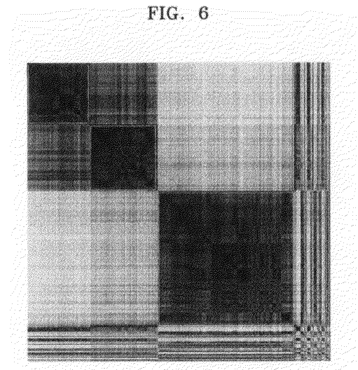

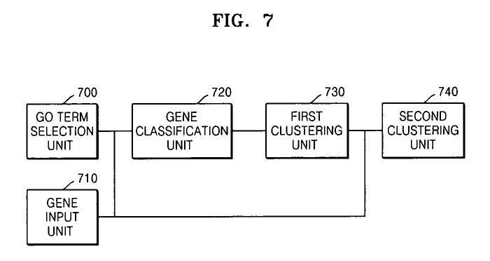

After the genes are rearranged, a similarity map is prepared by reflecting the sequence of the rearranged genes (Operation 220). The similarity map is used to support a user to determine blocks (seeds) of similarity. An example of the similarity map is illustrated in FIG. 6. Referring to FIG. 6, the brightness of each pair of two points (x, y) in the figure represents the similarity between the two data objects (two samples), i.e., x and y. The greater the similarity is, the darker the color of the points, and the smaller the similarity is, the lighter the color of the points. The similarity map is an embodiment of the present invention. The present invention can also use other similarity maps.

Once the similarity map is completed, a user set blocks of one or more genes that are considered to be similar to one another (Operation 230). Referring to FIG. 6, the selected gene blocks are shown in the shape of squares.

FIG. 3 is a flowchart illustrating a method of secondly clustering gene expression data sets according to an embodiment of the present invention. Referring to FIG. 3, the cluster obtained by the first clustering is the set of seeds for the second clustering (Operation 300). Centroids of each cluster are calculated from the seeds. There are various methods of setting the seeds by using the data sets, which can be applied to the present embodiment.

Each gene is allocated to the cluster (seeds of the cluster) having the highest similarity (Operation 310). The similarity can be calculated using the method that is adopted in the first clustering.

All the genes allocated to each cluster and the seed of the cluster may not have a satisfactory similarity. Therefore, if the similarity is lower than a designated similarity, the user excludes the gene from the cluster (Operation 320).

FIG. 7 is a block diagram of an apparatus for clustering gene expression profiles according to an embodiment of the present invention. Referring to FIG. 7, the apparatus for clustering the gene expression profiles comprises a GO term selection unit 700, a gene input unit 710, a gene classification unit 720, a first clustering unit 730, and a second clustering unit 740.

The GO term selection unit 700 displays the GO term tree on a screen to allow a user to select one or more GO terms. The GO term selecting unit 700 displays the GO term tree on a conventional GUI screen for user convenience, and receives a user's selection.

The gene input unit 710 receives gene expression data sets from a user. A preprocessing process of the gene expression data sets is obvious to one of ordinary skill in the art, and thus its detailed description will not be provided.

The gene classification unit 720 classifies genes of the gene expression data sets according to the selected GO terms.

The first clustering unit 730 measures a similarity between the genes allocated to each of the GO terms, rearranges the genes based on the similarity, and prepares a similarity map reflecting the order of the rearrangement. The first clustering unit 730 displays the similarity map on the screen to allow the user to set one or more blocks of the genes.

The second clustering unit 740 secondly clusters the genes by using the result of the first clustering unit 730 as seeds. In more detail, the second clustering unit 740 sets the results obtained from the first clustering unit 730 as a seed, allocates similar genes to each seed, and secondly clusters the genes. The second clustering unit 740 displays its result on the screen to allow the user to remove the genes having a lower similarity than a prespecified similarity from the cluster results.

The embodiments of the present invention can be written as computer programs and can be implemented in general-use digital computers that execute the programs using a computer readable recording medium. Examples of the computer readable recording medium include magnetic storage media (e.g., ROM, floppy disks, hard disks, etc.), optical recording media (e.g., CD-ROMs, or DVDs), and storage media such as carrier waves (e.g., transmission through the Internet). The computer readable recording medium can also be distributed network coupled computer systems so that the computer readable code is stored and executed in a distributed fashion.

The method of detecting a similar expression gene group by using the GO, according to the present invention effectively uses GO information when time-serial gene expression profile sets obtained from a micro array experiment are divided into clusters having similar expression patterns, thereby creating a biologically meaningful and highly reliable clustering result. The method can reduce information loss in GO seeds. Therefore, an effective study regarding a gene operation can be provided.

While the present invention has been particularly shown and described with reference to exemplary embodiments thereof, it will be understood by those of ordinary skill in the art that various changes in form and details may be made therein without departing from the spirit and scope of the invention as defined by the appended claims. The exemplary embodiments should be considered in a descriptive sense only and not for purposes of limitation. Therefore, the scope of the invention is defined not by the detailed description of the invention but by the appended claims, and all differences within the scope will be construed as being included in the present invention.

What is claimed is:

1. A method of clustering gene expression profiles comprising:
selecting one or more Gene Ontology (GO) terms from a GO tree;
receiving gene expression data sets;
classifying the gene expression data sets into groups according to the GO terms;
firstly clustering gene expression data belonging to each of the groups based on a similarity of the gene expression data; and
secondly clustering the gene expression data sets by using the result of the first clustering as a seed.

2. The method of claim 1, wherein the classifying of the gene expression data sets comprises:
allocating the gene expression data of the gene expression data sets to the groups of at least one or more related GO terms.

3. The method of claim 1, wherein the first clustering of the gene expression data comprises:
measuring a similarity between the gene expression data belonging to each group;
rearranging the gene expression data belonging to each group based on the similarity;
preparing a similarity map reflecting the rearranged gene expression data; and
setting at least one or more gene blocks having a similar expression pattern by using the similarity map.

4. The method of claim 3, wherein the measuring of the similarity comprises:
measuring the similarity between the gene expression data belonging to each group by using a Pearson correlation coefficient.

5. The method of claim 3, wherein the rearranging of the gene expression data comprises:
selecting any one piece of the gene expression data from the gene expression data belonging to each group, and arranging the other pieces of the gene expression data in a sequence of pieces most similar to the selected gene expression data.

6. The method of claim 1, wherein the second clustering of the gene expression data sets comprises:
setting a seed of each cluster obtained by the first clustering; and
clustering the gene expression data sets based on a similarity to the seed of each cluster.

7. The method of claim 6, further comprising: excluding the gene expression data having a similarity lower than a predetermined reference level from a result of the second clustering.

8. The method of claim 6, wherein the setting of the seed comprises: setting the seed by applying a centroid calculation of each cluster obtained by the first clustering.

9. An apparatus for clustering gene expression profiles comprising:
a GO selection unit selecting one or more GO terms from a GO tree;
a gene input unit receiving gene expression data sets;
a classification unit classifying the gene expression data sets into groups according to the GO terms;
a first clustering unit firstly clustering gene expression data belonging to each of the groups based on a similarity of the gene expression data; and
a second clustering unit secondly clustering the gene expression data sets by using the result of the first clustering as a seed.

10. The apparatus of claim 9, wherein the gene classification unit allocates the gene expression data of the gene expression data sets to the groups of at least one or more related GO terms.

11. The apparatus of claim 9, wherein the first clustering unit measures a similarity between the gene expression data belonging to each group, rearranges the gene expression data belonging to each group based on the similarity, prepares a similarity map reflecting the gene expression data, and sets at least one or more gene blocks having a similar expression pattern by using the similarity map.

12. The apparatus of claim 9, wherein the second clustering unit sets a seed of each clustering obtained from the first clustering unit and secondly clusters the gene expression data sets based on a similarity to the seed of each group.
